(19) United States
(12) Patent Application Publication
(10) Pub. No.: US 2025/0046435 A1
(43) Pub. Date: Feb. 6, 2025

(54) PREDICTING STRESS STATES

(71) Applicant: KONINKLIJKE PHILIPS N.V., EINDHOVEN (NL)

(72) Inventors: Joachim Dieter Schmidt, HAMBURG (DE); Thomas Erik Amthor, HAMBURG (DE); Tanja Nordhoff, HAMBURG (DE)

(21) Appl. No.: 18/716,974

(22) PCT Filed: Dec. 6, 2022
(86) PCT No.: PCT/EP2022/084486

§ 371 Date: Jun. 6, 2024

Related U.S. Application Data
(60) Provisional application No. 63/288,013, filed on Dec. 10, 2021.

(51) Int. Cl. G16H 40/20; A61B 5/00; A61B 5/11; A61B 5/16

(52) U.S. Cl. CPC G16H 40/20; A61B 5/1118; A61B 5/165; A61B 5/7275; A61B 2503/20

(57) ABSTRACT

In an embodiment, a method (100) is described. The method comprises receiving (102) information about an activity of a worker associated with an organization. The method further comprises predicting (104), using a prediction model, a stress state of the worker based on the information and a stress assessment provided by the worker. The method further comprises generating (106) an indication of whether a process for implementation by the organization is to be modified in view of the predicted stress state of the worker.

### TECHNICAL FIELD OF THE INVENTION

The invention relates to a method, a non-transitory machine-readable medium and apparatus for predicting stress state.

### BACKGROUND OF THE INVENTION

Workload is considered to be a source of stress and burnout for workers of an organization such as a hospital department. For example, workers such as clinicians associated with certain hospital departments may be subject to high workload owing to the nature of their work. By way of example, workers in radiology departments may experience significant amounts of stress given the clinical relevance of their work. An outcome of the work may be improved if stress could be eased, where such stress has been detected in the organization. An example way to determine worker stress situations and its root causes may involve engaging a specialist stress assessment service. Questionnaires and interviews may be used by such a stress assessment service to analyze stress and its roots. As part of this service, an analysis of the current stress situation in an organization may be carried out, followed by implementation of a change to how workers are managed, and followed by analysis of the outcome of implementing the change. The analyses may be carried out manually by specially trained consultants.

The manual analysis of stress may be expensive and may provide only a snapshot-like view at the problem of stress in an organization. An additional weakness of the manual approach is its limitation to a reasonable number of features that are regarded as root causes for stress. Many of these features correlate only slightly with situations that are perceived as stressful. Compounding the problem is the fact that different users may have different perception about stress.

### SUMMARY OF THE INVENTION

Certain aspects or embodiments described herein may relate to improving a process based on an improved prediction of stress of a worker. Certain aspects or embodiments may address problems relating to detected and/or compensating for detected stress in an organization.

In a first aspect, a method is described. The method is a computer-implemented method. The method comprises receiving information about an activity of a worker associated with an organization. The method further comprises predicting, using a prediction model, a stress state of the worker based on the information and a stress assessment provided by the worker. The method further comprises generating an indication of whether a process for implementation by the organization is to be modified in view of the predicted stress state of the worker.

Some embodiments relating to the first and other aspects are described below.

In some embodiments, the method comprises receiving information about the process. The information about the process comprises information about an asset associated with the organization for implementing at least part of the process. The indication of whether the process is to be modified comprises an indication of whether a change is to be made in terms of the asset used to implement at least part of the process.

In some embodiments, the information about the asset comprises information about at least one of: a role of the asset; and/or a capability and/or availability of the asset for carrying out the activity.

In some embodiments, the asset associated with the organization comprises at least one of: the worker: another worker associated with the organization; and/or a resource associated with the organization.

In some embodiments, the process comprises a workflow specifying a set of activities to be carried out by the organization as part of providing a service to a plurality of users of the service.

In some embodiments, the information about the activity of the worker comprises an indicator of the worker's stress state derived from activity data associated with the activity.

In some embodiments, the activity data comprises at least one of: a timestamp associated with the activity: a duration of the activity: an idle time of the worker and/or another worker with the same role as the worker before and/or after the activity: a number of parallel tasks of the worker and/or another worker with the same role as the worker for each activity carried out by the worker as part of the process: a total number of activities related to the worker and/or another worker with the same role as the worker as part of the process: a number of dependencies between the worker and other workers associated with the organization with the same or different roles to the worker: a previous process and/or activity in which the worker and/or another worker was involved: a type of the activity: a type of the process: a time of day associated with the activity: a day of week associated with the activity: a location associated with the activity; and/or a classification of a patient being cared for as part of the process.

In some embodiments, the prediction model comprises a machine learning model trained using the activity data associated with the worker and a ground truth label obtained from the stress assessment provided by the worker.

In some embodiments, the prediction model is configured to predict the stress state of the worker based on a role of the worker.

In some embodiments, the method further comprises modifying the process based on the generated indication. The modifying is based on an objective. The objective comprises at least one of: reducing the stress state of the worker and/or at least one other worker associated with the organization: reducing a cost of implementing the process; reducing a duration of implementing the process; and/or increasing throughput of the process.

In some embodiments, the process is modified based on a number of the objectives to be taken into account. The modification is configured to vary the process in order to meet a condition by varying at least one of: an order of a set of activities to be implemented as part of the process depending on predefined data specifying an allowed order: a choice of which worker of the worker and/or another worker associated with the organization is to be assigned to which of a set of activities associated with the process depending on predefined data specifying a capability and/or availability of the worker and/or the other worker: an assignment of a resource associated with the organization to the process; a proposed duration of an activity associated with the process where there is an opportunity to vary the duration by accelerating or omitting an unnecessary task associated with the activity; and/or a scheduling of a set of users of a service provided by the organization.

In some embodiments, modifying the process comprises causing a workflow engine to send a notification to a computing device associated with the organization. The notification is configured to cause the computing device to facilitate a change to the process based on the generated indication. The computing device is at least one of: associated with the worker and configured to provide an instruction for the worker based on the notification; associated with another worker and configured to provide an instruction for the other worker based on the notification: associated with a manager of the process for managing a set of assets associated with the organization and configured to cause an asset of the set of assets to implement the change based on the notification by sending an instruction to another computing device associated with the asset of the set of assets in order to implement the change; and/or associated with a resource of the organization and configured to control an operation of the resource based on the notification.

In some embodiments, the method comprises receiving input data, entered via an electronic interface associated with an admin of the organization, indicative of a prioritization of the objective to be taken into account when generating the indication of whether the process for implementation by the organization is to be modified in view of the predicted stress state of the worker. The objective to be taken into account comprises at least one of: the stress state of the worker and/or another worker associated with the organization; the cost of implementing the process: the duration of the process; and/or the throughput of the process.

In a second aspect, a non-transitory machine-readable medium is described. The non-transitory machine-readable medium stores instructions which, when executed by processing circuitry, cause the processing circuitry to implement the method of any of the first aspect and/or related embodiments.

In a third aspect, apparatus is described. The apparatus comprises processing circuitry communicatively coupled to an interface for receiving information about an activity of a worker associated with an organization. The apparatus further comprises a machine-readable medium storing instructions which, when executed by the processing circuitry, cause the processing circuitry to predict, using a prediction model, a stress state of the worker based on the information and a stress assessment provided by the worker. The instructions further cause the processing circuitry to generate an indication of whether a process for implementation by the organization is to be modified in view of the predicted stress state of the worker.

Certain aspects or embodiments described herein may provide various technical improvements in the context of, for example, identifying worker stress based on potentially massive amounts of data, identifying stress based on gathered data that does not directly indicate worker stress, mitigating or reducing such stress to improve the outcome of a process implemented by workers of the organization and/or facilitating an improved way to manage assets, including workers and/or resources, of an organization in order to improve the outcome of the process and/or reduce or mitigate stress.

These and other aspects of the invention will be apparent from and elucidated with reference to the embodiment(s) described hereinafter.

### BRIEF DESCRIPTION OF DRAWINGS

Exemplary embodiments of the invention will now be described, by way of example only, with reference to the following drawings, in which:

FIG. 1 refers to a method of predicting stress state according to an embodiment;

FIG. 2 is a schematic drawing of a system for predicting stress state according to an embodiment:

FIG. 3 refers to a method of predicting stress state according to an embodiment:

FIG. 4 is a schematic drawing of a user interface for managing a process implementation according to an embodiment:

FIG. 5 refers to a method of receiving information about a process according to an embodiment:

FIG. 6 refers to a method of modifying a process according to an embodiment:

FIG. 7 refers to a method of receiving input data according to an embodiment:

FIG. 8 is a schematic drawing of a machine-readable medium for implementing various embodiments; and

FIG. 9 is a schematic drawing of apparatus for implementing various embodiments.

### DETAILED DESCRIPTION OF THE EMBODIMENTS

As used herein, a “worker” may refer to a person working for an organization such as a hospital.

As used herein, an “activity of a worker” may refer to any activity carried out by the worker e.g., as part of their work duties. The term “activity” may refer to active or passive behavior of the worker. For example, a passive behavior such as an idle time of the worker or a break may still refer to an activity of the worker. An active behavior may refer to any task carried out by the worker e.g., as part of their work duties.

As used herein, a worker “stress state” may refer to a level of stress experienced by the worker. The stress state may or may not be quantifiable. For example, the stress state may be relative term that can be used to define a comparative stress of the worker at a certain time with respect to another time and/or with respect to another worker. This could be represented by a stress “score” e.g., on a sliding scale of stress. In another example, the stress state may refer a qualitative assessment of the worker's stress. In another example, the stress state may refer to a perceived stress of the worker. For example, different workers may experience stress in different ways (e.g., they may or may not be aware of the stress) and the throughput of their work may be increased or reduced, depending on various factors.

As used herein, a “process” may refer to a workflow used by an organization to carry out a function of the organization (such as providing a service). In the context of a clinical scenario such as in a hospital, the process may refer to how workers are assigned to tasks (e.g., in terms of which tasks they are assigned, at what time they are to perform those tasks and/or using which resources e.g., medical equipment, rooms, etc.). Thus, when a hospital has a set of patients to care for, the process may refer to how to manage the hospital's assets (including workers and/or medical equipment) in order to care for the set of patients. The process may be implemented manually (e.g., by a human admin), semi-automatically (e.g., computer-implemented with human “admin” input) or fully automatically (e.g., computer-implemented with no human input). In some cases, an organization may run multiple processes at any time and the worker may be involved in one or a plurality of these processes.

As highlighted above, it can be difficult to identify worker stress in an organization such as a hospital. There may be poor visibility or understanding of when workers are stressed. There may not be time to perform analysis and the stress situation may evolve over time. While stress may, for some people, lead to an increased workflow, this is no guarantee that the quality of the work being performed is optimum. In some cases, stress may lead to reduced throughput. In some cases, stress may have serious consequences on the quality of the work output, which could be risky in a hospital context. In a complex organization such as a hospital that implements a correspondingly complex process to manage its assets to thereby provide its service, it might be possible for a human admin to account for worker stress manually but the lack of visibility of stress throughout the organization may make this manual task difficult. Further, while workers could periodically self-assess their stress, such assessments may not be reliable and/or difficult for a human admin to interpret.

Various embodiments described below may address at least one such issue and/or various other issues.

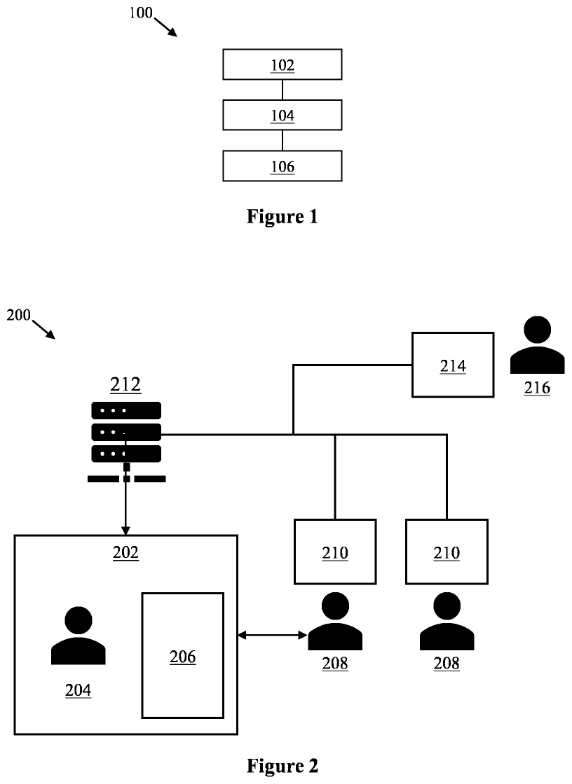

FIG. 1 refers to a method 100 of predicting stress state according to an embodiment. The method 100 is computer-implemented (e.g., by processing circuitry of a computing system that has access to certain data as described below). Details of the set-up of the computing system and other components for implementing various embodiments are described in more detail below.

The method 100 comprises, at block 102, receiving information about an activity of a worker associated with an organization.

The information may be provided in any appropriate format to be interpreted by the processing circuitry and may comprise content that is directly and/or indirectly indicative of the worker's stress state. In some cases, the information may have been collected by any computing device associated with the activity itself, the worker themselves or with which worker has interacted as part of the activity. Any data collected by such computing device(s) may be indicative of the worker's stress state, even if this is not directly evident from the data if such data was to be assessed by a human admin. For example, in the clinical context, the number of instances and/or time spent interacting with medical equipment may be indicative of the stress state. For example, multiple instances of interactions and/or too much/too little time being spent interacting may be indicative of worker stress. In some cases, the information comprises data produced by the computing devices such as a use report, diagnostics information or any other data that is indicative of what tasks have been carried out by the worker, how long they took or any other contextual data derived from the activity that might be usable for predicting stress.

The method 100 further comprises, at block 104, predicting a stress state of the worker based on the information and a stress assessment provided by the worker.

The stress assessment provided by the worker may comprise a subjective assessment of the worker's stress. In some cases, the stress assessment may allow the worker to submit a quantifiable (e.g., using sliding scales (e.g., from 1 to 10) in a questionnaire format where each question may directly or indirectly probe their stress with the answer representing a “cue” of their stress state) and/or qualitative record (e.g., a written or spoken description of their work, which may provide a “cue” of their stress state) of how they appear to perceive their stress at the time of completion. The stress assessment may be provided to the processing circuitry in an electronic format so that it can be interpreted by the processing circuitry. The stress assessment may be a historical record or an up-to-date stress assessment, and may be indicative of how the worker perceived or perceives their stress. In some cases, multiple stress assessments may be provided by the worker over time and these may be used for the prediction, where appropriate.

The stress state is predicted using a prediction model. The prediction model may be configured to examine cues from the “information” and the “stress assessment” in order to make its prediction. In some cases where the stress assessment comprises a qualitative record provided by the worker, the prediction model may use natural language processing to understand and/or interpret written or spoken description in the stress assessment. In some cases where the stress assessment comprises a quantitative record provided by the worker, the prediction model may incorporate any “scores” provided by the worker as part of its prediction.

The method 100 further comprises, at block 106, generating an indication of whether a process for implementation by the organization is to be modified in view of the predicted stress state of the worker.

In some cases, the indication may be used by a computer-implemented engine for controlling or managing the process(es) implemented by the organization such as a workflow engine (e.g., a software service or “engine” that provides the run time execution environment for a process instance. The workflow engine may provide operational functions to support the execution of (instances of) processes, based on the process definitions). The “indication” may be in any appropriate format so that it can be interpreted by the computer-implemented engine. Depending on whether the worker is stressed, the indication may cause the process to be modified to account for the stress or reduce the effect of the stress for the worker and/or for the overall process. On the other hand, if the worker is predicted to be not too stressed, they may be deployed for a certain part of the process to alleviate stress of other workers and/or improve implementation of the process.

A description of how the process might be modified, and how this may be implemented within the context of the organization, is given below.

Certain embodiments described herein (including the method 100 and other embodiments) may address at least one of various problems described herein, or any related problems, by implementing an (automatic/non-manual) analysis of a potentially massive number of features and other process data that may characterize the operation of an organization. In the context of an organization such as a hospital, such features may be obtained from electronic information systems such as a Radiology Information System (RIS), Hospital Information System (HIS), Picture Archiving and Communication System (PACS) and/or a workflow engine. At least one of the various sources of data may be leveraged in order to make predictions about the worker's stress state in such a way that would not normally be possible by a manual analysis of the data. Such predictions may identify stress that might otherwise be masked by the worker (e.g., since the available data from their activities may comprise cues which the worker cannot mask easily), more accurately identify stress and/or gain a better understanding of how stress is affecting the process and/or the organization as a whole.

In certain embodiments as described below, features from at least one of the sources of data may be used to train a prediction model implementing a machine learning (ML) model that uses the subjective stress level assessment of users as ground truth. Once trained, the ML model can detect and predict stress levels automatically, thus providing the basis for mitigations of any detected stress (e.g., via the indication described in relation to the method 100). A potential benefit of using a subjective stress assessment provided by the worker may be that the trained model may be capable of detecting stress in such a way that accommodates the different perception of stress within a workforce. For example, an objective stress assessment used to train the ML model may not be applicable to all workers and may provide inaccurate predictions, in some cases, as compared to using a subjective stress assessment.

Thus, certain embodiments described herein refer to implementations of the prediction model such as a machine-learning implementation. However, the stress prediction may be based on much more data than a manual admin can accommodate. A large variety of sources may provide the data in various formats where the data may or may not provide direct cues to the worker's stress state. A computer-implemented prediction model (whether ML-based or not ML-based) may be capable of handling large amounts of data (i.e., a “feature set”), such as may be generated in a complex organization such as a hospital, and make its prediction of the worker's stress state. The wide variety of available data in a complex organization may provide challenges for an admin to determine stress state based on all of this data. Use of the prediction model may facilitate a quicker, more accurate and/or more representative analysis of the worker's stress state since it may accommodate such a large amount of possible data when making its prediction.

Thus, the prediction may be based on a large feature set that includes not only the current activity of the worker, but also on other elements of the current process(es) and their interconnection. Thus, in some cases relating to a clinical scenario, the whole clinical and operational context can be used as a basis for the stress prediction. By way of example, tracking the worker and their activities across time based on, for example, the data generated by virtue of the worker carrying out tasks may provide the interconnection between the current process and other processes involving the worker.

While the method 100 refers to a process, the worker mentioned in the method 100 may or may not be part of the process at the time. For example, if the worker is stressed, they may be given time off/assistance (which is facilitated with the “indication” to cause a change to the process, as discussed below) to improve process implementation and/or reduce risks that may reduce the effectiveness of the process. If the worker is not stressed, they may be deployed (as specified via the “indication”) to take part in the process, for example, to alleviate stresses of other workers, improve process implementation and/or reduce risks that may reduce the effectiveness of the process.

A system for implementing various embodiments described herein, including method 100 and other embodiments, is now described.

FIG. 2 is a schematic drawing of a system 200 for predicting stress state according to an embodiment. The system 200 represents a clinical context although other contexts are possible, depending on the organization implementing the system 200. Thus, the process delivered by the system 200 relates to the provision of healthcare services where data is collected from various sources and used to predict stress (e.g., according to the method 100 and other embodiments described herein).

In the system 200, a patient context 202 refers to a patient 204 and medical equipment 206 associated with the patient. The process delivered by the system 200 provides services to the patient 204. As part of the system, a worker 208 such as a caregiver or clinician interacts with the patient context 202 (i.e., an interaction with the patient 204 themselves and/or the associated medical equipment 206). Two workers 208 are depicted in the system 200 although there may be one or more than two workers 208 involved in caregiving as part of the process at various times (e.g., due to the varying shift patterns, the various roles of the workers 208 and/or complex healthcare scenarios).

The workers 208 are associated with a computing device 210) (in this case, each worker 208 is associated with a computing device 210 although there may be more or fewer computing devices 210 per worker 208), which may allow the workers 208 to integrate with and/or facilitate the process. For example, the computing device 210 may allow the worker 208 to actively and/or passively input data such as information regarding their activities to facilitate running the process and/or receive data such as instructions on what tasks are to be carried out by worker 208. With reference to the method 100, in some cases, the computing device 210 may facilitate the input of the stress assessment by worker 208 and/or providing instructions to the worker 208 in accordance with certain embodiments described herein.

The system 200 further comprises a computing system 212 (e.g., comprising processing circuitry (not shown) for implementing certain embodiments described herein such as the method 100). The computing system 212 is communicatively coupled to sources of data such as the patient context 202 (e.g., via an interface with the medical equipment 206) and the computing devices 210. The computing system 212 may also have access to other data such as the RIS, HIS, PACS, etc. The computing system 212 may implement a workflow engine e.g., to facilitate/manage the process implemented by the organization.

The computing system 212 may be implemented locally (e.g., within the organization's locations) or remotely (e.g., via a cloud service or server implemented by the organization themselves or a third-party service provider). Thus, the collection of data may be performed locally where such data may be processed locally or elsewhere. The result of the processing may lead to a modification of the process, which may be indicated to any computing device associated with the system 200 in various manners such as described herein.

The computing system 212 is further communicatively coupled to a further computing device 214 associated with an admin 216 (e.g., a human or computer-based operative). The admin 216 may manage the operation of the computing system 212, input parameters such as information about new patients 204 received into the system 200 and/or control the implementation of certain parts of the process, etc.

As part of implementation of the system 200, the computing system 212 may collect data from various sources such as described above. Such data may be processed in order to implement the process. There may be a large amount of data to process, especially in complex scenarios such as the clinical context. The computing system 212 may continually or periodically analyze the data it has access to in order to determine how the process is to be implemented, e.g., based on parameters set by the admin 216. The computing system 212 may perform the stress prediction in accordance with the method 100 for each worker 208 e.g., based on their subjective stress assessment and/or any other data the computing system 212 has access to. The computing system 212 may generate the indication of whether a process for implementation by the organization is to be modified in view of the predicted stress state of the worker 208. Based on the generated indication, the computing system 212 may take certain action such as deploying assets such as the worker 208, another worker 208 and/or resources such as the medical equipment 206 or other facilities to appropriate parts of the organization e.g., in order to improve the implementation of the process to take into account the stress state of the worker(s) 208. Thus, the computing system 212 may optimize process(es) and/or resource allocation using the prediction as input for an objective function as described below.

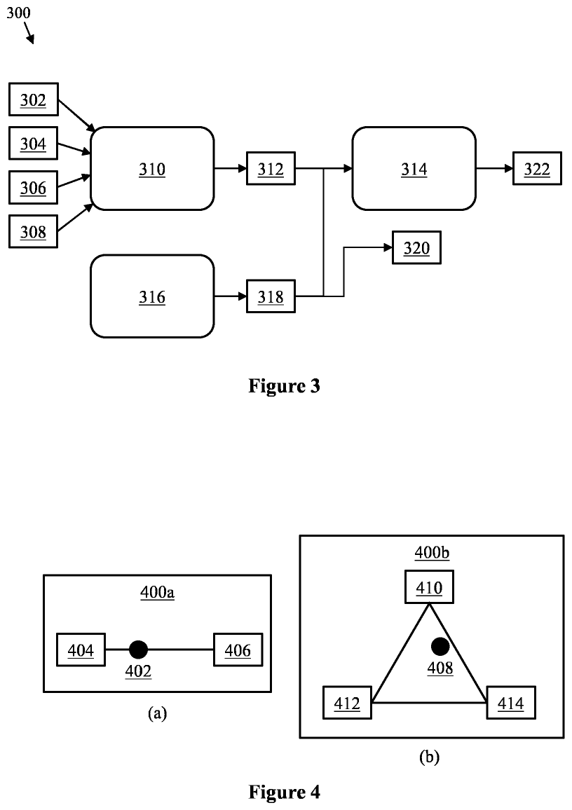

FIG. 3 refers to a method 300 of predicting stress state according to an embodiment. Reference is made to FIG. 2 in the description of the method 300. Various blocks of the method 300 are described below. Certain blocks may be omitted or implemented in a different order to that depicted by FIG. 3.

Some possible sources of data are represented by blocks 302, 304, 306 and 308. Block 302 refers to the RIS, Block 304 refers to the HIS. Block 306 refers to the PACS. Block 308 refers to workflow data provided by the workflow engine (i.e., the workflow data may indicate information about the process and the workflow engine may be implemented by the computing system 212). Other sources of data or a different combination of data sources may be called upon for the implementation of various embodiments described herein.

Any number or of combination of these sources of data may be called upon by a service application 310 implemented by the computing system 212. The service application 310 may facilitate the collection of the data from the various sources.

At block 312, the service application 310 may process the collected data. In some cases, this processing can involve formatting such data in a unified format, or at least providing the data in the appropriate format so that it can be interpreted for the purpose of the various embodiments described herein. The processing may comprise collating data, filtering the data, appending relevant information to the data, removing irrelevant information from the data, etc., so that the stress prediction can be performed, at block 314. Examples of such processing may include providing information on any of the worker 208 activities, the roles of such workers 208, any of the data provided by the service application 310 and/or appending relevant information such as a timestamp to certain data.

At block 316, the worker 208 submits their stress assessment (e.g., via an application running on their computing device 210). In addition to being indicative of their stress state, the stress assessment may comprise any further relevant information such as the worker's 208 role, a time stamp associated with the time of completion of the stress assessment, an identifier of the worker 208 within the organization. Such information may be provided by the worker 208 as part of their stress assessment or may be automatically populated by the computing system 212 and/or the application running on the worker's 208 computing device 210.

At block 318, the stress assessment (including any further relevant information) may be processed and submitted to the computing system 212 so that the stress prediction can be performed at block 314. In some cases, the stress assessment may be used, at block 320, in a training phase for a machine learning (ML) model associated with the stress prediction. In some cases, the training may be based on a training dataset provided by the same organization or a different organization or set of organizations where data collected from such organization(s) may be provided for the training dataset. In other words, the ML model may be initially trained and then deployed to an organization or the ML model may be trained within the organized to which it is deployed, or a hybrid of both scenarios.

Where ML is used, the ground truth (label) for learning may be provided by the respective role/worker 208 through their stress assessment provided at blocks 316/318. A ML model may be trained to interpret the information provided by the stress assessment in accordance with how the training was conducted. For example, if a qualitative assessment is provided and the ML model has been appropriately trained, the ML model may examine the stress assessment for indicators of stress and use this as part of its prediction. If a quantitative assessment is provided, which allows the worker 208 to indicative their stress via a number (e.g., a sliding scale value between 0 and 10, where 0 might mean lowest stress level and 10 might mean highest stress level), the ML model may use such a number as input to make its prediction.

At block 322, the worker's 208 stress state is output as a result of the stress assessment at block 314. In some cases, the stress state may further comprise any other information that may be relevant to the implementation of the process such as the worker's 208 role and/or their identifier. Although not shown, the output stress state may modify the process (to be) implemented by the workflow engine, which may then be fed back into a subsequent implementation of the method 300 in order to verify that the stress state has been reduced or that another objective has been or is expected to be achieved.

The time stamping of processed data may allow for the consideration of trends over time. For example, where machine learning (ML) is used for the prediction, the processed data may be provided as input vectors for the ML model implemented by the computing system 212, which may use time-stamped stress assessments as “ground truth” during the training phase.

The application for allowing the worker 208 to provide their stress assessment (as run on their computing device 210) may have a user interface that allows the worker 208 to submit their own assessment of their stress state. The other data collected (e.g., from the data sources 302 to 308) may provide “cues” about the worker's stress state. Where the computing system 212 implements ML-based stress prediction, the computing system 212 may output stress state data that can be used for mitigation (e.g., via the “indication” referred in the method 100).

Where ML is used, the ML model can be realized using any appropriate model such as decision trees, random forests, support vector machines, neural networks, etc.

The feature set provided to the ML model (e.g., as processed at blocks 312 and/or 318) may comprise quantities derived from factors relating to a process which may be linked to a certain role or worker 208, such as the activity duration, the idle time of the role/worker 208 before/after the activity, the number of parallel tasks of the role/worker 208 for each activity, the total number of activities related to the role/worker 208 in the process, the number of dependencies to other roles (e.g., how much different roles are needed in the implementation of the process), the type of activity, the type of process, time of day, day of week, classification of patient(s) involved in the process, a previous process and/or activity in which the worker and/or another worker was involved, a location associated with the activity, etc. Other factors may be relevant parts of the feature set. Any number or combination of these factors may be used by the ML model as part of its prediction.

Once trained, the ML model may output the expected/predicted stress state for the worker 208 for a specified process. Such a stress state may be useable for optimizing the process. For example, the workflow engine implemented by the computing system 212 may use the output of the ML model in order to optimize the process it is presently implementing or is to implement. In some cases, this may involve a different deployment of assets associated with the organization in such a way that takes into account the predicted stress state of the worker 208 (at the present time or in the future).

Thus, once the predicted stress is available for a process, this can be used to optimize process(es) (e.g., deploy assets/manage resources) in such a way that the total expected stress state for each worker 208 is minimized, or at least better managed than without the stress prediction.

In addition to the “lowest stress” objective, there may be other objectives, such as the total cost of the process or the total duration of the process. The latter two objectives may in some cases be calculated directly from the process definition without the need for a ML components if the duration of each activity is known and the resources allocated to each activity can be associated with a cost.

For a process, an objective function, ƒ=Σᵢ^{N}wᵢaᵢ may be defined, where N is the number of objectives to take into account, wᵢ is the relative weight of objective i, and aᵢ is the objective value. An optimization model may be employed to find the optimum variation of the processes, scheduling and/or resource allocation (all within given allowed ranges) in order to minimize the objective function. Possible choices for an optimization algorithm may be a simulated annealing type model, a genetic optimizer, or similar models. In order to minimize the objective function, the model may attempt to vary certain factors. These factors that can be varied may include, for example, the order of activities in the processes (e.g., where predefined conditions may sometimes allow changing the order), the choice of which staff member (worker 208) is assigned to which of several parallel activities (e.g., depending on predefined capabilities of the workers 208), the assignment of other resources (such as rooms or equipment) to the individual activities, the proposed duration of activities where there may be an option to vary the duration by accelerating or omitting unnecessary tasks, the scheduling of patients throughout the day and/or the scheduling of staff (e.g., varying the number, type and/or role of workers 208 available at a certain time).

To implement such an objective function, the weights may be normalized such that Σᵢwᵢ=1.

FIG. 4 is a schematic drawing of user interfaces 400a, 400b for managing a process implementation according to an embodiment. Parts (a) and (b) of FIG. 4 represent different possible ways to implement the user interface 400a,b. The user interface 400a,b may be implemented by (e.g., a graphical user interface of) the computing device 214 associated with the (human) admin 216 (e.g., a clinical department head). The admin 216 may make a decision about their organization's priorities or preferences at a certain time and may use the user interface to input their decision e.g., by changing the relative weighting to achieve various objectives such as reducing stress, increasing throughput and/or reducing cost (e.g., more workers cost more). The computing system 212 may receive the input and then modify the process should this be needed in view of the admin's 216 decision/preference.

The change of weighting may be realized by providing a slider on the user interface 400a,b that can be moved between e.g., two or three targets on a screen, as exemplified in FIG. 4.

In FIG. 4(a), a 1-dimensional slider 402 can be moved (e.g., using a touchscreen or a mouse pointer) on a line between two objectives (e.g., a lower stress level 404 or higher throughput 406). The relative weights can then be defined as the relative distance to the opposite objective along the depicted line. A higher throughput might increase stress in the organization but might be needed in times of urgency such as the arrival of multiple patients at the same time. However, if stress states become too high, the admin 216 might indicate that a lower stress level is needed. In either case, the workflow engine can work out how to implement the process in a manner specified by the admin 216, and may cause the deployment of assets accordingly.

In FIG. 4(b), a 2-dimensional slider 408 can be moved (e.g., using a touchscreen or a mouse pointer) within a triangle, where the vertices of the triangle represent three different objectives (i.e., lower stress level 410, higher throughput 412 and lower cost 414). In this case, the relative weights may be defined as the relative distances of the slider to the triangle edge opposite of the vertex representing the respective objective.

In some cases, the impact of implementing the proposed variations (e.g., as input by the admin 216) may be retrospectively analyzed by comparing the expected change of the objectives with the actual change of the objectives. This may involve the use of periodic feedback from workers 208 about the experienced stress level so that this can be fed back into the prediction, to retrain or rework the stress prediction model.

In some cases, when the admin 216 selects the optimization objectives, the proposed variations leading to a minimization of the objective function may be presented to the admin 216, via the user interface 400a,b, to be acknowledged or rejected. This can be done within the same user interface 400a,b used to select the optimization objectives. If rejected by the admin 216, the optimization model implemented by the workflow engine can proceed to propose another slightly less optimal solution for the process.

Some embodiments relating to the above are now described.

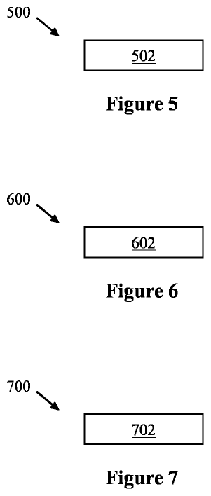

FIG. 5 refers to a method 500 of receiving information about a process according to an embodiment. The method 500 may be implemented by processing circuitry such as implemented by the computing system 212 of FIG. 2, to which reference is made in the description given below. The method 500 may refer to information useable in relation to the certain embodiments described herein such as the method 100. In this regard, the indication provided by the method 100 may depend on the method 500.

The method 500 comprises, at block 502, receiving information about the process. Such information may be received from the workflow engine. The information about the process may comprise information about an asset associated with the organization for implementing at least part of the process. The indication of whether the process is to be modified comprises an indication of whether a change is to be made in terms of the asset used to implement at least part of the process.

In some cases, the “change” may refer to replacing an existing asset (such as the worker 208, who might be stressed), deploying an additional asset (such as the worker 208 themselves and/or another worker 208) or making further facilities available as part of the process. Thus, based on the received information, the process may be modified (by the workflow engine) in accordance with the indication provided by the method 100.

In some embodiments, the information about the asset comprises information about a role of the asset and/or a capability and/or availability of the asset for carrying out the activity. For example, if an asset is available for deployment to implement a part of the process, such an asset may be deployed to alleviate stress of a worker 208.

In some embodiments, the asset associated with the organization comprises at least one of: the worker 208: another worker 208 associated with the organization; and/or a resource. In some cases, the resource could refer to physical resource such as medical equipment, a room, property, etc., accessible to/owned by the organization, etc. The resource could also refer to a virtual resource such as a computing resource e.g., for performing data analysis associated with providing care to a patient.

In some embodiments, the process comprises a workflow specifying a set of activities to be carried out by the organization as part of providing a service to a plurality of users of the service. In the context of a clinical scenario, a “user” may refer to a patient. The set of activities in this context may refer to medical tasks such as monitoring, treatment and/or other care to be provided to the patient. Thus, the workflow may specify which workers are to perform which task at which time and/or using which resources available to the organization at to be used in order to implement the workflow.

In some embodiments, the information about the activity of the worker comprises an indicator of the worker's 208 stress state derived from activity data associated with the activity. The activity data may provide direct or indirect hints about the worker's 208 stress state.

In some embodiments, the activity data comprises at least one of: a timestamp associated with the activity: a duration of the activity: an idle time of the worker 208 and/or another worker 208 with the same role as the worker before and/or after the activity: a number of parallel tasks of the worker 208 and/or another worker 208 with the same role as the worker for each activity carried out by the worker 208 as part of the process (e.g., a higher burden may be placed on some workers 208 even though they have the same role): a total number of activities related to the worker 208 and/or another worker 208 with the same role as the worker as part of the process: a number of dependencies between the worker 208 and other workers 208 associated with the organization with the same or different roles to the worker 208: a previous process and/or activity in which the worker and/or another worker was involved: a type of the activity: a type of the process: a time of day associated with the activity: a day of week associated with the activity: a location associated with the activity; and/or a classification of a patient being cared for as part of the process.

Any number or combination of these factors may represent activity data that may directly infer or indirectly hint at the worker's stress state.

In the case of a ML model that is trained to identify elevated stress, the ML model may detect patterns from certain activity data that is indicative of elevated stress. Some indicators may be clear to a human admin 216 such as the worker 208 not having much idle time (which in some cases could be indicative of there being too much work). However, other more subtle indicators may not be so clear to a human admin 216 such as historical indicators such as previous activities that a worker 208 was involved with and/or whether or not the working environment is noisy, which might be inferred from the location. Thus, by leveraging the “overall picture” provided by the various data sources 302 to 308 and the subjective stress assessment provided at block 316, the ML model may detect patterns that are not otherwise detectable to a human admin 216 due to the amount of data generated by data collection/lack of easy interpretation.

Thus, in some embodiments, the prediction model comprises an ML model trained using the activity data associated with the worker and a ground truth label obtained from the stress assessment provided by the worker. In some cases, the ML model may be trained using activity data from a set of workers with the same role/different roles, as well as their stress assessments.

In some embodiments, the prediction model is configured to predict the stress state of the worker 208 based on a role of the worker 208. For example, the prediction model may be able to take into account the worker's 208 role when predicting stress of the worker 208. For example, some roles may be associated with a higher stress level.

FIG. 6 refers to a method 600 of modifying a process according to an embodiment. The method 600 may be implemented by processing circuitry such as implemented by the computing system 212 of FIG. 2, to which reference is made in the description given below. The method 600 may refer to how the indication provided by the method 100 may be used (i.e., by the workflow engine to modify the process, where needed). In other words, the method 600 may be implemented by the workflow engine, as implemented by the computing system 212.

The method 600 comprises, at block 602, modifying the process based on the generated indication, wherein the modifying is based on an objective (e.g., the objective function described above). The objective comprises at least one of: reducing the stress state of the worker 208 and/or at least one other worker 208 associated with the organization: reducing a cost of implementing the process: reducing a duration of implementing the process; and/or increasing throughput of the process.

In some embodiments, the process is modified based on a number of the objectives to be taken into account. The modification is configured to vary the process in order to meet a condition by varying at least one of: an order of a set of activities (e.g., tasks for workers 208 of the organization) to be implemented as part of the process depending on predefined data specifying an allowed order: a choice of which worker of the worker 208 and/or another worker 208 associated with the organization is to be assigned to which of a set of activities associated with the process depending on predefined data specifying a capability and/or availability of the worker 208 and/or the other worker 208 (e.g., it may be better to deploy a less stressed worker 208): an assignment of a resource associated with the organization to the process: a proposed duration of an activity associated with the process where there is an opportunity to vary the duration by accelerating or omitting an unnecessary task associated with the activity; and/or a scheduling of a set of users of a service provided by the organization.

Thus, any appropriate variation may be made to the process in order to meet the condition. In some cases, the condition may be to minimize the objective function, “f”, as described above. An asset such as a worker 208 or resource may be deployed at an appropriate time at an appropriate location to respond to demand placed on the organization. The workflow engine may recognize such demand and respond accordingly, e.g., by modifying the process and ensuring that the assets are deployed e.g., to meet the condition.

In some embodiments, modifying the process comprises causing the workflow engine to send a notification to a computing device associated with the organization. The notification may be configured to cause the computing device to facilitate a change to the process based on the generated indication.

In some cases, the computing device may be associated with the worker 208 (e.g., their computing device 210) and configured to provide an instruction for the worker 208 based on the notification (e.g., via its user interface).

In some cases, the computing device may be associated with another worker 208 and configured to provide an instruction for the other worker 208 (e.g., via their computing device 210) based on the notification.

In some cases, the computing device may be associated with a manager of the process (e.g., the computing device 214 associated with the admin 216) for managing a set of assets associated with the organization and configured to cause an asset of the set of assets to implement the change based on the notification by sending an instruction to another computing device associated with the asset of the set of assets in order to implement the change.

In some cases, the computing device may be associated with a resource of the organization (e.g., a computing device implemented by the medical equipment 206) and configured to control an operation of the resource based on the notification.

In a possible scenario, if there are two workers 208 that showed different stress response in activities that are now scheduled in parallel (e.g., education of patient A and reporting of results to patient B), the workflow engine may decide to swap the assignment of tasks to decrease the overall stress perception of both workers 208. Such a swap may be indicated to both workers 208 via their respective computing devices 210.

In some cases, the notification may be useable to control a computing device 210 such that it operates to implement the modified process e.g., by managing the associated worker 208 via a user interface to inform the worker 208 about the received notification and/or controlling any other device in any way to inform the asset (worker 208 or a resource) that the process has been or is to be modified and that the asset is to operate differently as a result of the modification.

In other similar words, there may be circumstances where the notification may modify the operation of the computing device 210 and/or the equipment 206 in such a way that the process is modified to account the predicted stress state of the worker 208. For example, the computing device 210 may inform the worker 208 that they are to rest or do another task. In another example, the computing device 210 may provide different options to the worker 208 and/or cause them to do tasks in a different order. In another example, the computing device 210 may change its interaction with the worker 208 in order to take into account the worker's stress (e.g., more/fewer notifications, checking on the worker 208, etc.). In another example, a resource such as medical equipment may be deployed/activated in response to the notification. In another example, the notification may cause the cancellation of a scheduled patient altogether (e.g., where the computing device 210 schedules such a patient) if the overall stress level of the worker 208 is predicted to rise above a certain limit.

FIG. 7 refers to a method 700 of receiving input data according to an embodiment. The method 700 may be implemented by processing circuitry such as implemented by the computing system 212 of FIG. 2, to which reference is made in the description given below. The method 700 may refer to the use of the input data provided by the user interface depicted by FIG. 4. In other words, the method 700 may be implemented by the workflow engine, as implemented by the computing system 212, based on the input provided by the user interface such as depicted by FIG. 4.

The method 700 comprises, at block 702, receiving input data, entered via an electronic interface (e.g., interface 400a,b) associated with an admin 216 of the organization. The received input data is indicative of a prioritization of the objective to be taken into account when generating the indication of whether the process for implementation by the organization is to be modified in view of the predicted stress state of the worker. The objective to be taken into account comprises at least one of: the stress state of the worker and/or another worker associated with the organization (e.g., either attempt to meet the objective of reducing stress or allowing the stress to increase if another objective is more relevant at a certain time): the cost of implementing the process (e.g., increase or decrease cost depending on the priority); the duration of the process (e.g., increase or decrease the duration depending on the priority); and/or the throughput of the process (e.g., increase or decrease the throughput depending on the priority).

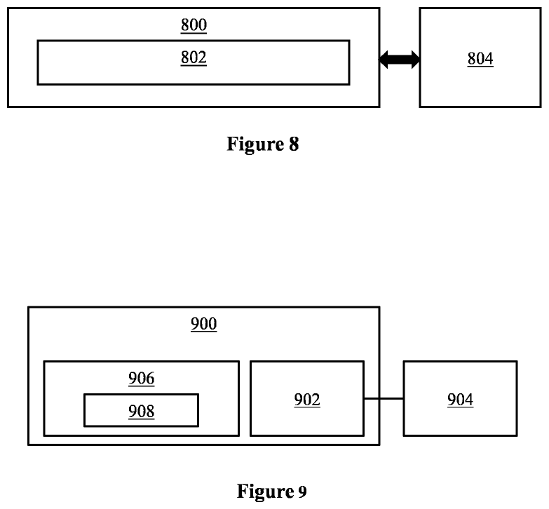

FIG. 8 is a schematic drawing of a non-transitory machine-readable medium 800 for implementing various embodiments described herein. The machine-readable medium 800 stores instructions 802 which, when executed by processing circuitry 804, cause the processing circuitry 804 to implement the method of any of the embodiments described herein (e.g., the method 100, 500, 600, 700 or any related methods). The machine-readable medium 800 and/or the processing circuitry 804 may be implemented by the computing system 212 of FIG. 2.

FIG. 9 is a schematic drawing of apparatus 900 for implementing various embodiments described herein. The apparatus 900 may be implemented by the computing system 212 of FIG. 2.

The apparatus 900 comprises processing circuitry 902. The processing circuitry 902 is configured to communicate with an interface 904. The interface 904 may be any interface (wireless or wired) implementing a communications protocol to facilitate exchange of data with other devices such as the equipment 206 and/or computing devices 210, 214. In this case, the interface 904 may receive information about an activity of a worker associated with an organization in accordance with block 102 of the method 100.

The apparatus 900 further comprises a machine-readable medium 906 (e.g., non-transitory or otherwise) storing instructions 908 which, when executed by the processing circuitry 902, cause the processing circuitry 902 to implement various embodiments described herein (e.g., method 100 or any of the associated embodiments). In this regard, the instructions 908 cause the processing circuitry 902 to predict, using the prediction model, a stress state of the worker 208 based on the information and a stress assessment provided by the worker 208 (e.g., in accordance block 104 of the method 100). The instructions 908 further cause the processing circuitry 902 to generate an indication of whether a process for implementation by the organization is to be modified in view of the predicted stress state of the worker 208 (e.g., in accordance block 106 of the method 100).

In some embodiments, the instructions 908 may comprise further instructions to cause the processing circuitry 902 to implement the functionality of any embodiments relating to the method 100.

Methods, machine-readable media and apparatus described herein may be implemented at the computing system 212. However, other devices such as the computing devices 210, 214 described in the system 200 may assist with the implementation of such methods, machine-readable media and apparatus (e.g., due to sending/receiving data to/from the computing system 212).

While the invention has been illustrated and described in detail in the drawings and foregoing description, such illustration and description are to be considered illustrative or exemplary and not restrictive: the invention is not limited to the disclosed embodiments.

One or more features described in one embodiment may be combined with or replace features described in another embodiment.

Embodiments in the present disclosure can be provided as methods, systems or as a combination of machine-readable instructions and processing circuitry. Such machine-readable instructions may be included on a non-transitory machine (for example, computer) readable storage medium (including but not limited to disc storage, CD-ROM, optical storage, flash storage, etc.) having computer readable program codes therein or thereon.

The present disclosure is described with reference to flow charts and block diagrams of the method, devices, and systems according to embodiments of the present disclosure. Although the flow charts described above show a specific order of execution, the order of execution may differ from that which is depicted. Blocks described in relation to one flow chart may be combined with those of another flow chart. It shall be understood that each block in the flow charts and/or block diagrams, as well as combinations of the blocks in the flow charts and/or block diagrams can be realized by machine readable instructions.

The machine-readable instructions may, for example, be executed by a general-purpose computer, a special purpose computer, an embedded processor, or processors of other programmable data processing devices to realize the functions described in the description and diagrams. In particular, a processor or processing circuitry, or a module thereof, may execute the machine-readable instructions. Thus, functional modules of apparatus and other devices described herein may be implemented by a processor executing machine readable instructions stored in a memory, or a processor operating in accordance with instructions embedded in logic circuitry. The term ‘processor’ is to be interpreted broadly to include a CPU, processing unit, ASIC, logic unit, or programmable gate array etc. The methods and functional modules may all be performed by a single processor or divided amongst several processors.

Such machine-readable instructions may also be stored in a computer readable storage that can guide the computer or other programmable data processing devices to operate in a specific mode.

Such machine-readable instructions may also be loaded onto a computer or other programmable data processing devices, so that the computer or other programmable data processing devices perform a series of operations to produce computer-implemented processing, thus the instructions executed on the computer or other programmable devices realize functions specified by block(s) in the flow charts and/or in the block diagrams.

Further, the teachings herein may be implemented in the form of a computer program product, the computer program product being stored in a storage medium and comprising a plurality of instructions for making a computer device implement the methods recited in the embodiments of the present disclosure.

Elements or steps described in relation to one embodiment may be combined with or replaced by elements or steps described in relation to another embodiment. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems.

1. A computer-implemented method, comprising:
receiving information about an activity of a worker associated with an organization;
predicting, using a prediction model, a stress state of the worker based on the information and a stress assessment provided by the worker; and
generating an indication of whether a process for implementation by the organization is to be modified in view of the predicted stress state of the worker,
wherein the information about the activity of the worker comprises an indicator of the worker's stress state derived from activity data associated with the activity, and
wherein the prediction model comprises a machine learning model trained on the activity data associated with the worker and a ground truth label obtained from the stress assessment provided by the worker.

2. The method of claim 1, further comprising receiving information about an asset associated with the organization for implementing at least part of the process, and wherein the indication of whether the process is to be modified comprises an indication of whether a change is to be made in terms of the asset used to implement at least part of the process.

3. The method of claim 2, wherein the information about the asset comprises information about at least one of:
a role of the asset; and/or
a capability and/or availability of the asset for carrying out the activity.

4. The method of claim 2, wherein the asset associated with the organization comprises at least one of:
the worker;
another worker associated with the organization; and/or
a resource associated with the organization.

5. The method of claim 1, wherein the process comprises a workflow specifying a set of activities to be carried out by the organization as part of providing a service to a plurality of users of the service.

6. (canceled)

7. The method of claim 1, wherein the activity data comprises at least one of:
a timestamp associated with the activity;
a duration of the activity;
an idle time of the worker and/or another worker with the same role as the worker before and/or after the activity;
a number of parallel tasks of the worker and/or another worker with the same role as the worker for each activity carried out by the worker as part of the process;
a total number of activities related to the worker and/or another worker with the same role as the worker as part of the process;
a number of dependencies between the worker and other workers associated with the organization with the same or different roles to the worker;
a previous process and/or activity in which the worker and/or another worker was involved;
a type of the activity;
a type of the process;
a time of day associated with the activity;
a day of week associated with the activity;
a location associated with the activity; and/or
a classification of a patient being cared for as part of the process.

8. (canceled)

9. The method of claim 1, wherein the prediction model is configured to predict the stress state of the worker based on a role of the worker.

10. The method of claim 1, comprising:
modifying the process based on the generated indication, wherein the modifying is based on an objective, wherein the objective comprises at least one of:
reducing the stress state of the worker and/or at least one other worker associated with the organization;
reducing a cost of implementing the process;
reducing a duration of implementing the process; and/or
increasing throughput of the process.

11. The method of claim 10, wherein the process is modified based on a number of the objectives to be taken into account, and wherein the modification is configured to vary the process in order to meet a condition by varying at least one of:
an order of a set of activities to be implemented as part of the process depending on predefined data specifying an allowed order;
a choice of which worker of the worker and/or another worker associated with the organization is to be assigned to which of a set of activities associated with the process depending on predefined data specifying a capability and/or availability of the worker and/or the other worker;
an assignment of a resource associated with the organization to the process;
a proposed duration of an activity associated with the process where there is an opportunity to vary the duration by accelerating or omitting an unnecessary task associated with the activity; and/or
a scheduling of a set of users of a service provided by the organization.

12. The method of claim 10, wherein modifying the process comprises causing a workflow engine to send a notification to a computing device associated with the organization, wherein the notification is configured to cause the computing device to facilitate a change to the process based on the generated indication, wherein the computing device is at least one of:
associated with the worker and configured to provide an instruction for the worker based on the notification;
associated with another worker and configured to provide an instruction for the other worker based on the notification;
associated with a manager of the process for managing a set of assets associated with the organization and configured to cause an asset of the set of assets to implement the change based on the notification by sending an instruction to another computing device associated with the asset of the set of assets in order to implement the change; and/or
associated with a resource of the organization and configured to control an operation of the resource based on the notification.

13. The method of claim 10, comprising receiving input data, entered via an electronic interface associated with an admin of the organization, indicative of a prioritization of the objective to be taken into account when generating the indication of whether the process for implementation by the organization is to be modified in view of the predicted stress state of the worker, wherein the objective to be taken into account comprises at least one of:
the stress state of the worker and/or another worker associated with the organization;
the cost of implementing the process;
the duration of the process; and/or
the throughput of the process.

14. A non-transitory machine-readable medium storing instructions which, when executed by processing circuitry, cause the processing circuitry to implement the method of claim 1.

15. An apparatus, comprising:
processing circuitry communicatively coupled to an interface for receiving information about an activity of a worker associated with an organization; and
a machine-readable medium storing instructions which, when executed by the processing circuitry, cause the processing circuitry to:
predict, using a prediction model, a stress state of the worker based on the information and a stress assessment provided by the worker; and
generate an indication of whether a process for implementation by the organization is to be modified in view of the predicted stress state of the worker,
wherein the information about the activity of the worker comprises an indicator of the worker's stress state derived from activity data associated with the activity, and
wherein the prediction model comprises a machine learning model trained on the activity data associated with the worker and a ground truth label obtained from the stress assessment provided by the worker.

16. The apparatus of claim 13, wherein information is received about an asset associated with the organization for implementing at least part of the process, and wherein the indication of whether the process is to be modified comprises an indication of whether a change is to be made in terms of the asset used to implement at least part of the process.

17. The apparatus of claim 16, wherein the information about the asset comprises information about at least one of:
a role of the asset; and/or
a capability and/or availability of the asset for carrying out the activity.

18. The apparatus of claim 16, wherein the asset associated with the organization comprises at least one of:
the worker;
another worker associated with the organization; and/or
a resource associated with the organization.

19. The apparatus of claim 15, wherein the process comprises a workflow specifying a set of activities to be carried out by the organization as part of providing a service to a plurality of users of the service.

20. The apparatus of claim 15, wherein the prediction model is configured to predict the stress state of the worker based on a role of the worker.

21. The apparatus of claim 15, wherein the processing circuitry is configured to modify the process based on the generated indication and based on an objective, wherein the objective comprises at least one of:
reduce the stress state of the worker and/or at least one other worker associated with the organization;
reduce a cost of implementing the process;
reduce a duration of implementing the process; and/or
increase throughput of the process.

22. The apparatus of claim 21, wherein the process is modified based on a number of the objectives to be taken into account, and wherein the modification is configured to vary the process in order to meet a condition by varying at least one of:
an order of a set of activities to be implemented as part of the process depending on predefined data specifying an allowed order;
a choice of which worker of the worker and/or another worker associated with the organization is to be assigned to which of a set of activities associated with the process depending on predefined data specifying a capability and/or availability of the worker and/or the other worker;
an assignment of a resource associated with the organization to the process;
a proposed duration of an activity associated with the process where there is an opportunity to vary the duration by accelerating or omitting an unnecessary task associated with the activity; and/or
a scheduling of a set of users of a service provided by the organization.
